(19) United States
(12) Patent Application Publication
(10) Pub. No.: US 2015/0201902 A1
(43) Pub. Date: Jul. 23, 2015

(54) DUAL-MODALITY ENDOSCOPE, METHOD OF MANUFACTURE, AND USE THEREOF

(71) Applicant: THE UNIVERSITY OF CONNECTICUT, Farmington, CT (US)

(72) Inventors: Quing Zhu, Mansfield, CT (US); Patrick Kumavor, Southbridge, MA (US)

(21) Appl. No.: 14/413,823

(22) PCT Filed: Jun. 27, 2013
(86) PCT No.: PCT/US2013/048119

§ 371 Date: Jan. 9, 2015

Related U.S. Application Data
(60) Provisional application No. 61/670,347, filed on Jul. 11, 2012.

(51) Int. Cl. A61B 8/00; A61B 8/12; A61B 1/00; A61B 1/273; A61B 1/303; A61B 1/31; A61B 1/307; A61B 5/00; A61B 1/233

(52) U.S. Cl. CPC A61B 8/4416; A61B 5/0095; A61B 8/12; A61B 1/00165; A61B 1/0011; A61B 1/233; A61B 1/303; A61B 1/31; A61B 1/307; A61B 1/2733

(57) ABSTRACT

An endoscope includes a sheath; an ultrasound transducer disposed in the sheath to transmit an ultrasound frequency and to receive an image signal comprising an ultrasound signal and photoacoustic signal; and a plurality of optical fibers interposed between the sheath and ultrasound probe to transmit light; wherein the sheath comprises: a first end configured to accept the ultrasound transducer and plurality of optical fibers; and a second end to pass the ultrasound frequency and light out of the sheath. A process to make the endoscope comprises shaping a material to form a sheath; inserting an ultrasound transducer into the sheath; disposing a plurality of optical fibers into the sheath; and coupling an end of the sheath to the ultrasound transducer. A system for imaging comprises an endoscope; a near-infrared light source coupled endoscope; and an acquisition device to acquire an image signal from the endoscope.

### STATEMENT REGARDING FEDERALLY SPONSORED RESEARCH

This invention was made with government support under Grant No. 1R01CA151570 awarded by National Cancer Institute of the National Institutes of Health. The government has certain rights in the invention.

### BACKGROUND

Biological imaging of living tissue involves radiation from the X-ray region through the microwave region of the electromagnetic spectrum. Techniques such as computed tomography (CT) and magnetic resonance imaging (MRI) provide a glimpse into structural features of tissue, and mathematical processing of two-dimensional data can render three-dimensional images of such tissue. Both hard and soft tissue can be imaged. Contrast agents allow improved resolution and enhancement of images as well as a means for imaging of cavities. For example, micro bubble contrast agents have been used in echocardiograms for cardiac shunt detection.

Imaging with non-ionizing radiation is preferred due to concerns over tissue damage. Further, many practitioners and patients seek to alleviate risk factors associated with certain contrast agents. However, some widely used imaging techniques have resolution insufficient to discover lesions and tumors at the on-set of growth. The art is always receptive to materials or methods that have enhanced resolution and image quality and that are also rich in information content.

### SUMMARY

Disclosed herein is an endoscope comprising: a sheath; an ultrasound transducer disposed in the sheath to transmit an ultrasound frequency and to receive an image signal comprising an ultrasound signal and photoacoustic signal; and a plurality of optical fibers interposed between the sheath and ultrasound probe to transmit light; wherein the sheath comprises: a first end configured to accept the ultrasound transducer and plurality of optical fibers; and a second end to pass the ultrasound frequency and light out of the sheath.

Also disclosed herein is a process of making an endoscope comprising: shaping a material to form a sheath; inserting an ultrasound transducer into the sheath; disposing a plurality of optical fibers into the sheath; and coupling an end of the sheath to the ultrasound transducer.

Further disclosed is a system for imaging comprising: an endoscope comprising: a sheath; an ultrasound transducer disposed in the sheath; and a plurality of optical fibers interposed between the ultrasound transducer and the sheath; a near-infrared light source coupled to the plurality of optical fibers; and an acquisition device to acquire an image signal from the ultrasound transducer.

The above described and other features are exemplified by the following figures and detailed description.

### BRIEF DESCRIPTION OF THE DRAWINGS

Referring now to the figures, which are embodiments, and wherein like elements are numbered alike:

FIG. 1 is a cross-section of an embodiment of an endoscope;

FIG. 2 is a view from the bottom of the endoscope of FIG. 1;

FIG. 3 is a photograph of an embodiment of an endoscope from one perspective;

FIG. 4 is photograph of an embodiment of a sheath;

FIG. 5 is a photograph of an embodiment of a coupling member;

FIG. 6 is a cross-section of another embodiment of an endoscope;

FIG. 7 shows various distributions of optical fibers with respect to a ultrasound transducer array;

FIG. 8 is a schematic drawing of an embodiment of an imaging system;

FIG. 9 shows simulated illumination distributions at various depths within a model tissue for 2 optical fibers distributed about an ultrasound transducer;

FIG. 10 shows simulated illumination distributions at various depths within a model tissue for 6 optical fibers distributed about an ultrasound transducer;

FIG. 11 shows simulated illumination distributions at various depths within a model tissue for 18 optical fibers distributed about an ultrasound transducer;

FIG. 12 shows simulated illumination distributions at various depths within a model tissue for 36 optical fibers distributed about an ultrasound transducer;

FIG. 13 is a photoacoustic image of a tube of blood;

FIG. 14 is a photoacoustic image of the tube shown in FIG. 13 covered by a layer of chicken breast tissue;

FIG. 15 is a co-registered image of a human ovary; and

FIG. 16 is a co-registered image of the human ovary shown in FIG. 15 covered by a layer of chicken breast tissue.

### DETAILED DESCRIPTION

It has been found that an endoscope for dual-modality imaging exhibits high sensitivity, resolution, optical contrast, and frame rate. Moreover, the endoscope is non-destructive to tissue and useful in non-invasive or minimally invasive procedures. In addition, the endoscope, while useful in in vivo (e.g., a tissue, a tissue cavity, or an organ) of a subject, also can be used external to a subject's body or even ex vivo. The endoscope herein therefore can improve diagnostic accuracy, patient comfort, and tissue analysis.

In photoacoustic imaging, a temporally short light pulse irradiates tissue in a target area with the light being absorbed by a chromophore in the tissue. Absorption of the light creates a transient temperature increase and subsequent thermoelastic expansion of the tissue, which generates an acoustic wave. An ultrasound transducer detects the acoustic wave. Acquired waveforms of emitted acoustic waves are used to reconstruct the optical absorption distribution of the chromophores in the tissue. An optical signature of biological tissue is the absorption of near infrared (NIR) radiation. Such absorption is related to blood hemoglobin content. Abnormally high growth rate of cancerous cells requires an elevated supply of nutrients and oxygen compared with normal, healthy tissue. Growth of the cancerous cells is sustained by increased vascularization of the tissue due to rapid growth of blood vessel networks or angiogenesis. The endoscope herein can image tumor angiogenesis development and allows detection of early stage tumor growth and cancer formation.

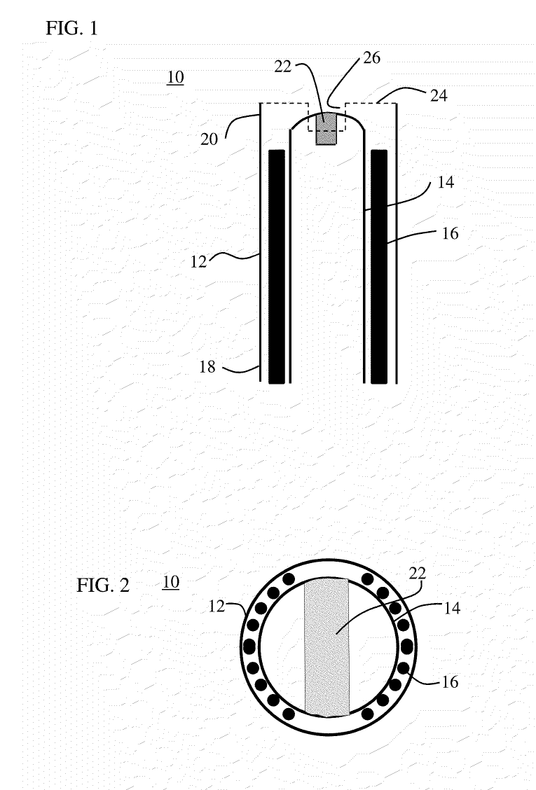

FIG. 1 shows a cross-section of an embodiment of an endoscope 10 that includes a sheath 12 with an ultrasound transducer 14 disposed in the sheath 12. A plurality of optical fibers 16 is interposed between the sheath 12 and ultrasound probe 14 to transmit light. The sheath 12 serves as a housing for the ultrasound transducer 14 and optical fibers 16. The ultrasound transducer 14 transmits an ultrasound frequency and receives an image signal, which includes an ultrasound signal (sometimes referred to as an ultrasound pulse echo) and photoacoustic signal. The sheath 12 includes a first end 18 configured to accept the ultrasound transducer 14 and plurality of optical fibers 16 and a second end 20 to pass the ultrasound frequency and light out of the sheath 12.

FIG. 2 is a view from the second end of the endoscope 10. An active region 22, e.g., an array of transducer elements, of the ultrasound transducer 14 is configured to transmit the ultrasound frequency and also to receive the image signal. The image signal, i.e., the ultrasound signal and photoacoustic signal, is received from a sample subjected to the light from the optical fibers 16 and ultrasound frequency from the ultrasound transducer 14. Thus, the endoscope is an imaging device that is configured to operate with dual-modalities.

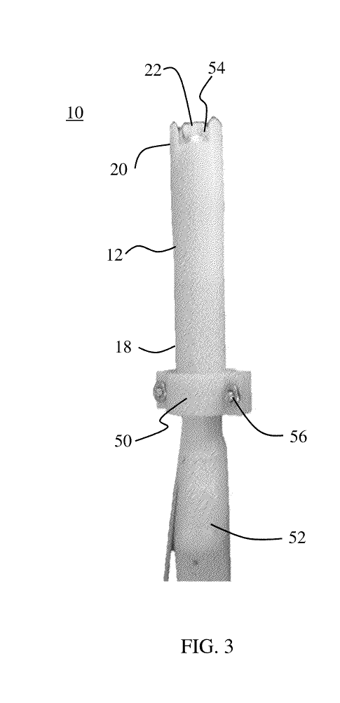

FIG. 3 is a photograph of an embodiment of the endoscope 10. In the embodiment, the endoscope 10 has a coupling member 50 that couples the sheath 12 to the ultrasound transducer 14 and plurality of optical fibers (not visible in FIG. 3). Additionally, the endoscope 10 can have a handle 52 disposed at the first end 18. A fastener 56, e.g., a screw, bolt, nut, rivet, staple, adhesive, and the like, can be used to secure the coupling member 50 to the endoscope 10. In some embodiments, the handle 52 and the ultrasound transducer 14 are an integrated member. In another embodiment, the sheath 12, handle 52, and ultrasound transducer 14 are an integrated member as a monolithic structure.

FIG. 3 shows that a tip 54 containing active region 22 of the ultrasound transducer 14 can be exposed by the sheath 12 in certain embodiments. The sheath 12 can be a shape that is effective to allow insertion of the endoscope 10 into a cavity, e.g., a vagina or rectum of a subject such as a human or animal. A transverse cross-sectional shape of the sheath 12 can be round, ellipsoidal, rectangular, triangular, and the like, or an truncated version thereof. The outer surface of the sheath 12 can be straight, tapered, or a combination thereof. Moreover, the sheath 12 can contain various provisions such as an opening, slit, protrusion, or undulation, e.g., along its length or at the first end 18 or second end 20.

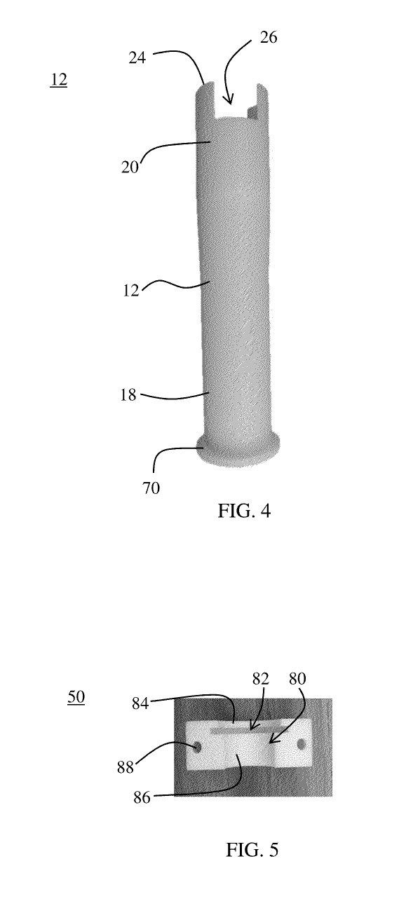

As shown in FIG. 4, the sheath 12 has a straight outer surface that extends from the first end 18 to the second end 20. An edge 24 (also shown as the dotted feature in FIG. 1) of the second end 20 can be completely co-planar or can have various shapes such as a shape which includes a notch 26. The notch 26 can be rectangular, curved, semi-circular, and the like. In an embodiment, the second end 20 has multiple notches 26. In a particular embodiment, the second end 20 has two notches 26 to pass the ultrasound frequency from the ultrasound transducer 14 from beyond the sheath 12 without obstruction. The first end 20 of the sheath 12 can terminate as a straight section or can have another shape or feature. In an embodiment, the first end 20 can include a rim 70. The rim 70 can protrude from an outer surface of the first end 20 to engage the coupling member 50. In an embodiment, the coupling member 50 can be an item that has an inner surface to mate with or receive the sheath 12, the ultrasound transducer 14, handle 52, or a combination comprising at least one of the foregoing. FIG. 5 is a photograph of a portion of the coupling member 50 having a surface 80, e.g., an inner surface, that mates with the rim 70 at inset 82, the first end 18 of the sheath 12 at first collar 84, and the ultrasound transducer 14 at second collar 86. The coupling member 50 mates with another coupling member to capture and secure the sheath 12 and ultrasound transducer 14 between the two coupling members 50 that are held together by a fastener 56 (as in FIG. 3) that can pass through hole 88 of the coupling member 50.

The sheath 12 encloses the optical fibers 16 and ultrasound transducer 14. Further, the sheath 12 protects these components as well as provides a guard against contacting tissue with the optical fibers 16 or body of the ultrasound transducer 14. Beyond protection, the relative position of the optical fibers 16 and ultrasound transducer 14 can be selected by their position inside the sheath 12. Thus, in an embodiment, the sheath 12 can be press fit over the combination of optical fibers 16 and ultrasound transducer 14. In another embodiment, the sheath 12 allows movement of the optical fibers 16, ultrasound transducer 14, or a combination thereof. Such motion can include rotary mobility, axial mobility, or a combination thereof. Rotary mobility includes rotation of the optical fibers 16 or ultrasound transducer 14 either about its body axis or about an axis within the sheath 12. Axial mobility includes longitudinal motion (e.g., retraction or extension of the optical fibers 16 or ultrasound transducer 14) in the sheath 12. In yet another embodiment, the ultrasound transducer 14 or optical fibers 16 are immobilized in the sheath 12. In a further embodiment, the ultrasound transducer 14 and optical fibers 16 have independent mobility from one another. In an alternative embodiment, the ultrasound transducer 14 and optical fibers 16 have synchronous mobility. In one embodiment, an optical fiber can move independently of another optical fiber. Alternatively, the motion of the optical fibers 16 is synchronous.

According to an embodiment, an inner surface of the sheath 12 facing the optical fibers 16 reflects the light from the optical fibers 16. In an embodiment, the inner surface can be made from or coated with a material that reflects the light, the material comprising or consisting of, e.g., a metal such as aluminum, silver, gold, platinum, copper, tin, tantalum, zinc, zirconium, silicon, an oxide thereof, or a combination thereof, e.g., an alloy. In an embodiment, the material reflects a near infrared wavelength.

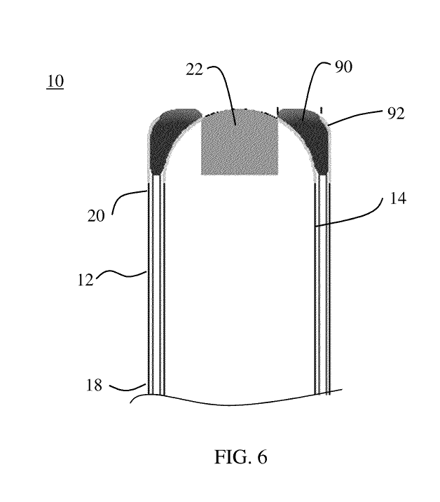

In an embodiment, an optic is disposed at the second end 20 of the sheath 12 and can transmit the light from the optical fibers 16, as in FIG. 6. The optic 90 can be any shape such as a shape that allows uniform diffusion of the light from optical fibers 16 onto a sample, e.g., tissue. The optical fibers 16 can be disposed to minimize a gap between the optical fibers 16 and the optic 90. In an embodiment, an optical cement or fluid can be used to match the refractive indexes of the optical fibers 16 and optic 90 to reduce an insertion loss. The optic 90 can be any material that transmits near infrared wavelengths. In an embodiment, the optic can be a quartz, a glass, a polymer such polymethylmethacrylate, and the like. In a non-limiting embodiment, the optic 90 can have a coating 92 that is a reflective material such as gold or silver to reflect the light from the optical fibers 15 so that the sheath 12 has high transmission efficiency for light from the optical fibers 16.

The sheath 12 can be made of a material that is strong enough to contain the optical fibers 16 and the ultrasound transducer 14. The material is further selected to be compatible with tissues, particularly when it is intended to be used in vivo. Exemplary materials include plastic, ceramic, glass, metal, or a combination thereof. In a particular embodiment, the sheath 12 is a metal such as stainless steel, nickel, aluminum, and the like; a plastic such as acrylonitrile-butadiene-styrene, polyurethane, polyimide, and the like; or a combination thereof. In certain embodiments, the sheath is flexible such that the endoscope can bend. In another embodiment, the sheath includes an articulation about which the endoscope bends or flexes. Thus, in some embodiments, the endoscope can bend in response to an applied force. The force can be a force transmitted from an external location to the endoscope. Alternatively, the force can be applied, for example, by pushing the endoscope against an object such as tissue.

It is contemplated that the uniformity of light at a sample, e.g., tissue, from the endoscope 10, is determined by the number of optical fibers as well as their distribution, size, position relative to the edge 24 of sheath 12, position relative to active area 22 of the ultrasound transducer 14, and the like. The position of the optical fibers 16 within the sheath 12 can be selected or adjusted such that the optical fibers 16 do not extend beyond the second end 20. According to an embodiment, the optical fibers 16 are recessed inside the sheath 12 such that optical fibers do not directly contact tissue. In some embodiments, the optical fibers 16 have a covering disposed on a surface of the optical fibers to protect tissue from contact with the optical fibers 16. In a particular embodiment, the covering is a film (e.g., a polymer such as polytetrafluoroethylene (Teflon)) disposed on the optical fibers. Thus, a barrier (e.g., the sheath or covering on the optical fibers) can separate the optical fibers from direct contact with tissue when the endoscope is in use.

Since the fluence of the light at tissue affects parameters such as imaging contrast as well as potential radiation damage of the tissue, the power level of the light from the fiber optics is selected and adjustable. In some embodiments, the power of the light and diffusion and thus uniformity of the light propagating from the optical fibers 16 and the endoscope 10 can be controlled by selection of the distance from the ends of the optical fibers 16 to the edge 24 of the second end 20 of the sheath 12. In a specific embodiment, the optical fibers 16 terminate from 0.1 millimeters (mm) to 15 mm before the second end 20 of the sheath 12, specifically 1 mm to 12 mm, and even more specifically 5 mm to 9 mm. In a further embodiment, the ultrasonic transducer 14 is disposed inside the sheath 12 to terminate before the edge 24 of the second end 20.

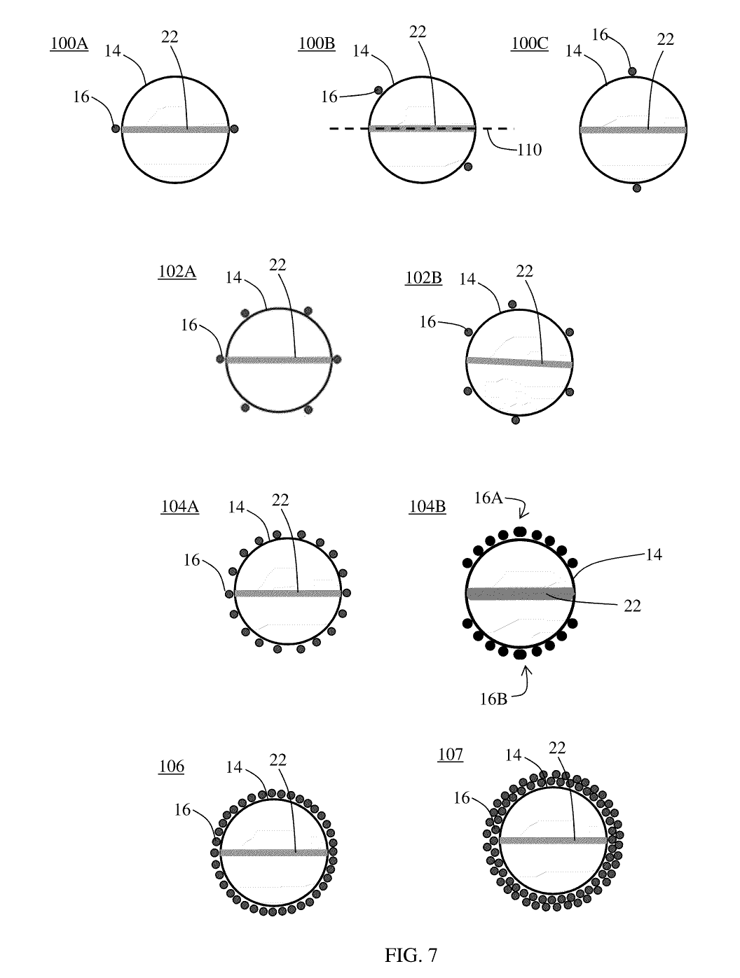

In an embodiment, the optical fibers 16 are perimetrically distributed about the ultrasound transducer 14 as in FIG. 1, which illustrates the distribution of 18 optical fibers 16. Any number of optical fibers 16 can be disposed in the sheath 12, including from 1 to 100 optical fibers, specifically 6 to 48 optical fibers, and more specifically 9 to 36 optical fibers. FIG. 7 shows several configurations of varying numbers of optical fibers 16 distributed about ultrasound transducer 14, such as a 2-optical fiber distribution (100A, 100B, 100C), 6-optical fiber distribution (102A, 102B), 18-optical fiber distribution (104A and 104B), and 36-optical fiber distribution 106. The optical fibers 16 can be distributed symmetrically or asymmetrically about the ultrasound transducer 14. In some embodiments, the optical fibers 16 can be positioned in more than one layer on the ultrasound transducer as in layers 112A, 112B in multilayer distribution 108. Although two layers are shown in FIG. 7, more than two layers of optical fibers 16 are envisioned as well. Additionally, the layers of optical fibers can be discontinuous so that there is a gap between neighboring optical fibers in the same layer of optical fibers.

In an embodiment, the optical fibers can be separated in two or more groups of optical fibers 16A, 16B as in the 36-optical fiber distribution 104B. The optical fibers 16 can be oriented with respect to the active area 22 of the ultrasound transducer 14 such that an optical fiber is coincident with an axis 110 of the active area 22. In some embodiments the optical fibers are not aligned with the active area 22 as in 2-optical fiber distributions (100B, 100C) and 6-optical fiber distribution 102B. For convenience of description, an angle of 0° corresponds to the right end of the active area 22, with angular measure increasing in a counter-clockwise direction such that 90° corresponds to the top optical fiber in the 2-optical fiber distribution 102C. In some embodiments, the closest fibers between two groups of optical fibers (see distribution 104B) can be separated by a selected distance, e.g., the width of the active area 22 of the ultrasound transducer 14, which in some embodiments corresponds to a width traversed by an output (e.g., the ultrasound frequency) of the ultrasound transducer 14.

The optical fibers 16 can be various sizes, i.e., have various diameters, including a diameter from 25 micrometers (μm) to 300 μm, specifically 50 μm to 250 μm, and more specifically 50 μm to 200 μm. The optical fibers can be made of an optical material that transmits, e.g., near infrared light with high efficiency. In an embodiment, the light from the optical fibers 16 is from 600 nanometers (nm) to 2000 nm, specifically 700 nm to 1000 nm, and mores specifically 700 nm to 900 nm. In an embodiment, the light transmitted by the optical fibers 16 has a uniform illumination distribution at a distance from 1 mm to 75 mm beyond the second end 20 of the endoscope 10, specifically 1 mm to 50 mm, and more specifically 5 mm to 40 mm. In a particular embodiment, a fluence of the light in the uniform illumination distribution has a Gaussian distribution. In another embodiment, the light from the optical fibers can be effected by, e.g., an optic 90 (FIG. 6) to shape the light into various non-Gaussian fluence profiles. The power of the light from the endoscope can be from 1 milliJoules per square centimeter (mJ/cm²) to 60 mJ/cm², specifically 1 mJ/cm² to 40 mJ/cm², and more specifically 1 mJ/cm² to 24 mJ/cm². According to an embodiment, the power of the light is equal to or less than a damage threshold of biological tissue, such as 24 mJ/cm².

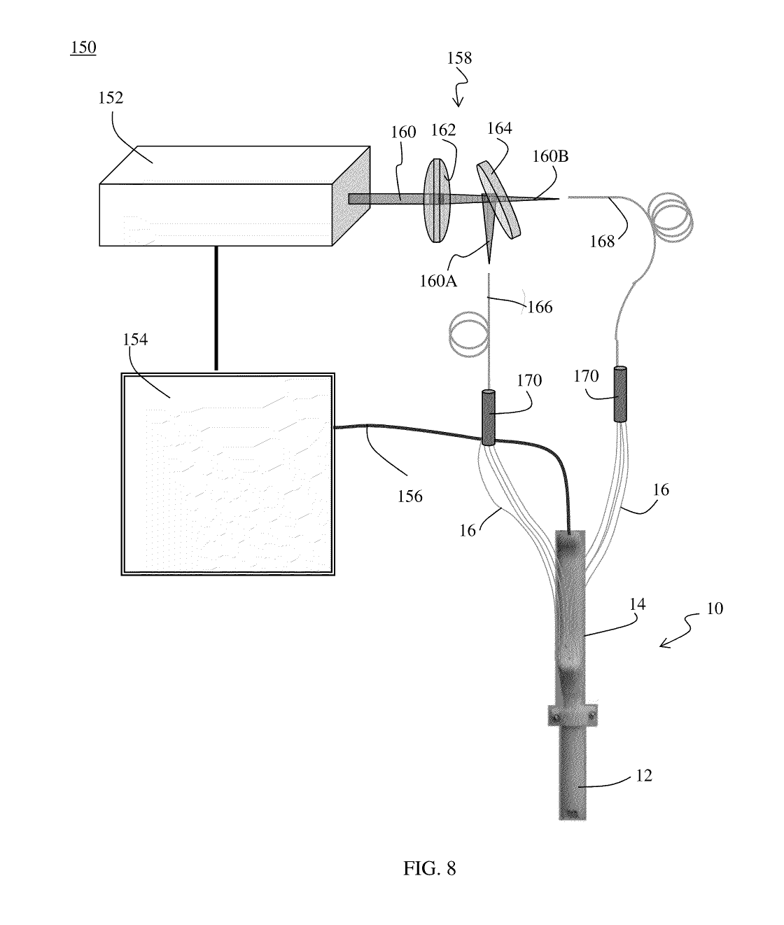

According to an embodiment, as shown in FIG. 8, a system for imaging includes an endoscope 10 that has a sheath 12, an ultrasound transducer 14 disposed in the sheath, and a plurality of optical fibers 16 interposed between the ultrasound transducer 14 and the sheath 12. A light source 152, e.g., near infrared light source, is coupled to the optical fibers 16, and an acquisition device 154 acquires an image signal from the ultrasound transducer 14 via signal line 156. The image signal contains, e.g., interleaved photoacoustic signals and ultrasound signals, which respectively correspond to photoacoustic waves emitted from tissue subjected to the near infrared light transmitted by the optical fibers 16 and ultrasound echoes from the tissue after probing with the ultrasound frequency emitted by the ultrasound transducer 14.

The system also includes an optical train 158 that couples the light 160 emitted from the light source 152 into the plurality of optical fibers 16. The optical train 158 can include various optics such as a neutral density filter, color filter, prism, lenses, and the like. In an embodiment, the optical train 158 has a convex lens 162 to focus the light 160 from the light source 152. Additionally, a beam splitter 164 is inserted into a path of the light 160 to split the light 160 into two beam paths. According to an embodiment, light 160A is directed onto a first optical fiber 166, and light 160B is directed onto a second optical fiber 168. First and second optical fibers are coupled to beam splitters 170, which split and direct the light into the optical fibers 16. Thus, the light 160 can be split from one beam into a plurality of beams that is directed into the plurality of optical fibers 16. In an embodiment, the beam splitter 170 can be, e.g., a 1×19 beam splitter wherein a single light beam 160A is input into the optical fiber 166 and further split by beam splitter 170 into additional beams (here 19 beams) of light corresponding to the number optical fibers 16 coupled to optical fiber 166 via beam splitter 170.

The light source 152 can be various light sources including a flash lamp, continuous light source, or laser. The light can be modulated either within the light source 152 or by an external element such a light chopper, including a rotary wheel or tuning fork, to produce a pulsed light beam. The pulse length of the light can be from 1 nanosecond (ns) to 500 ns, specifically 5 ns to 250 ns, more specifically 5 ns to 100 ns, and even more specifically 5 ns to 40 ns. The repetition rate of the light source can be 1 hertz (Hz) to 20 kilohertz (kHz), specifically 1 Hz to 1 kHz, and more specifically 1 Hz to 20 Hz. The repetition rate is selected depending on factors such as the photoacoustic decay time, data acquisition or processing delays, and the like. The light source can be for example a laser such as a Ti:sapphire laser pumped by an appropriate laser excitation source, e.g., an Nd:YAG laser. Although a peak power of the laser could potentially damage the input surface of the optical fibers, the laser power can be decreased by lowering the output power of the laser or insertion of optics in the beam path before the optical fiber, e.g., insertion of a neutral density filter, beam splitter, color filter, and the like.

The acquisition system 154 interacts with the light source 154 and ultrasound transducer 14. In an embodiment, a transient image signal, e.g., acoustic signals received by the ultrasound transducer 14 from a sample subjected to radiation from the endoscope 10 is transmitted to the acquisition system 154 for image processing and display. The acquisition system 154 can function in real time. The acquisition system can include, e.g., four 16-channel modules that are combined to form a 64-channel system. Each module can be controlled by a separate field-programmable gate array (FPGA) processor that can include analog and digital circuitry for acquisition, processing, and control of components of the imaging system 150. The FPGA can control the ultrasound transmission and detection, photoacoustic data acquisition process, parallel processing and storage of light beam information, and real-time switching between the two modalities, i.e., photoacoustic imaging and ultrasound imaging. Data storage of each module can be available for a digital signal processor (DSP) to access using an external memory interface (EMIF). Image reconstruction can be accomplished using a delay-and-sum algorithm. Ultrafast reconfiguration of the FPGA allows it to quickly switch between the two imaging modes (ultrasound and photoacoustic), perform transmission control, laser synchronization, internal memory structuring, beamforming, and EMIF structure memory sizing. As a result, the imaging system 150 can perform seamless co-registered photoacoustic imaging interlaced with ultrasound imaging.

The endoscope herein can be used for various non-invasive or minimally invasive procedures involving human or animal subjects. Thus, the endoscope can be used as a transvaginal probe, transrectal probe, transnasal probe, transesophageal probe, or transurethral probe since the endoscope is configured to be inserted into a tissue, cavity, or a combination comprising at least one of the foregoing. In another embodiment, the endoscope can be used ex vivo, as well as with living or dead biological tissue or biological tissue mimics. The endoscope herein has advantageous properties such as high resolution. The resolution is at least 0.1 mm, specifically 0.2 mm, and more specifically 0.5 mm, based on the ability to discern two features in an image acquired from the photoacoustic signal of tissue irradiated by the endoscope. In an embodiment, the image signals acquired by the endoscope are co-registered photoacoustic signals and ultrasound echo signals that correspond to tissue structure, vascularization, or a combination comprising at least one of the foregoing. Consequently, the endoscope can detect the early onset of cancer, e.g., ovarian cancer, and may extend the life of patients by allowing for more effective treatment options.

In an embodiment, the endoscope can be manufactured by shaping a material to form a sheath, inserting an ultrasound transducer into the sheath, disposing a plurality of optical fibers into the sheath, and coupling an end of the sheath to the ultrasound transducer. The sheath can be made by shaping a polymer material that includes three-dimensional printing, molding, thermoforming, and the like. In another embodiment, the sheath can be a metal that is subjected to powder processing, forging, casting, and the like. The sheath can be further machined and designed to a selected tolerance to mate with the combination of ultrasound transducer and optical fibers. Machining can include cutting, milling, lathing, lapping, and the like.

Set forth below are some embodiments of the endoscope, the process for making the endoscope, and the system for imaging with the endoscope disclosed herein.

Accordingly, an endoscope comprises a sheath; an ultrasound transducer disposed in the sheath to transmit an ultrasound frequency and to receive an image signal comprising an ultrasound signal and photoacoustic signal; and a plurality of optical fibers interposed between the sheath and the ultrasound transducer to transmit light, wherein the sheath comprises: a first end configured to accept the ultrasound transducer and plurality of optical fibers; and a second end to pass the ultrasound frequency and light out of the sheath. The endoscope further comprises a coupling member to couple the sheath to the ultrasound transducer and the plurality of optical fibers; a handle disposed at the first end wherein the handle and the ultrasound transducer are an integrated member; or an optic disposed at the second end to transmit the light from the plurality of optical fibers. With regard to the endoscope, the ultrasound transducer is disposed inside the sheath to terminate before an edge of the second end; the plurality of optical fibers terminates from 1 mm to 12 mm before the second end of the sheath; the endoscope is flexible such that the endoscope bends in response to an applied force; the ultrasound transducer or plurality of optical fibers is immobilized in the sheath, or the ultrasound transducer or plurality of optical fibers has a rotary mobility, axial mobility, or a combination comprising at least one of the foregoing in the sheath; an inner surface of the sheath comprises a coating effective to reflect the light from the plurality of optical fibers wherein the coating comprises aluminum, silver, gold, platinum, copper, tin, tantalum, zinc, zirconium, silicon, an oxide thereof, or a combination comprising at least one of the foregoing; the second end of the sheath comprises a notched structure configured to transmit the ultrasound frequency from the ultrasound transducer; the sheath comprises a metal; the plurality of optical fibers are perimetrically distributed about the ultrasound transducer; the optical fibers are divided into at least two groups of optical fibers, the two groups being separated by at least a distance corresponding to a width traversed by an output of the ultrasound transducer; the plurality of optical fibers includes more than 2 optical fibers; each of the optical fibers of the plurality of optical fibers has a diameter from 50 μm to 250 μm; the endoscope further comprises a film disposed on the plurality of optical fibers to prevent direct contact of the plurality of optical fibers with a tissue in use; a fluence of the light transmitted by the plurality of optical fibers has a uniform illumination distribution at a distance from 4 mm to 40 mm beyond the second end of the endoscope such that wherein the uniform illumination distribution is a Gaussian distribution; the light has a power from 1 mJ/cm² to 24 mJ/cm²; the light has a wavelength from 600 nm to 900 nm; the light has a pulse length from 5 ns to 200 ns; the light has a repetition rate from 1 Hz to 1 kHz; the endoscope is a transvaginal probe, transrectal probe, transnasal probe, transesophageal probe, or transurethral probe; or a resolution of the endoscope is at least 0.2 mm.

In another embodiment, a process of making the endoscope comprises shaping a material to form a sheath; inserting an ultrasound transducer into the sheath; disposing a plurality of optical fibers into the sheath; and coupling an end of the sheath to the ultrasound transducer to make the endoscope. A system for imaging comprises the endoscope that comprises: a sheath, an ultrasound transducer disposed in the sheath to transmit an ultrasound frequency and to receive an image signal comprising an ultrasound signal and photoacoustic signal, and a plurality of optical fibers interposed between the sheath and the ultrasound transducer to transmit light; a near-infrared light source coupled to the plurality of optical fibers; and an acquisition device to acquire an image signal from the ultrasound transducer, wherein the sheath comprises: a first end configured to accept the ultrasound transducer and plurality of optical fibers; and a second end to pass the ultrasound frequency and light out of the sheath. The system optionally comprises an optical train to couple the near-infrared light source to the plurality of optical fibers, wherein the optical train includes a lens to focus a light from the near-infrared light source onto an input end of the plurality of optical fibers or a beam splitter to split a light from the near-infrared light source into at least two light paths. The image signal comprises a photoacoustic signal and ultrasound signal.

The endoscope and imaging system comprising the endoscope are further illustrated by the following examples, which are non-limiting.

### EXAMPLES

### Example 1

### Monte Carlo Simulation of Light Beam Fluence

Monte Carlo (MC) analysis was used to simulate light fluence and uniformity delivered by the endoscope. A gynecological anatomy requires light to first penetrate through approximately 1 cm vaginal muscle wall before reaching an ovary during in vivo photoacoustic imaging. Consequently, a semi-infinite two-layer scattering medium with a planar boundary was used for the Monte Carlo simulation. A first layer of tissue was modeled as a vaginal muscle wall of 1 cm thickness and muscle tissue absorption coefficient of μₐ=0.03 wavenumbers (cm⁻¹) and reduced scattering coefficient of μₛ′=4 cm⁻¹. Soft tissue optical properties of μₐ=0.02 cm⁻¹ and μₛ′=8 cm⁻¹ were used as the second tissue layer, which represented an ovary in the model. A refractive index of the vaginal muscle was matched to the external environment near a simulated endoscope except at the location of the face of the endoscope, which was assigned an effective reflection coefficient of 0.7. Photon random walks were modeled by statistically sampling the probability distributions of the step size and angular deflection following an exponential and Henyey-Greenstein phase functions, respectively. During the simulation, a photon with unity weight (w) was introduced into a turbid medium from a distal end of a model optical fiber and then moves one step in a direction defined initially by the numerical aperture of the optical fiber. In a subsequent step, the step size and the scattering angle are chosen statistically. For each scattering event, a fraction of the photon weight, w×(μₐ/(μₛ+μₐ)), was deposited at each location, with μₐ and μₛ denoting the absorption and scattering coefficients respectively. A photon continues to scatter until it was transmitted from the boundaries or its weight decreased to a value that was less than a threshold value. The photon sample size was varied, but no less than 1 million photons were used in each simulation. Accumulated photon weights at each grid element corresponded to the absorbed energy. Finally, the fluence was obtained by dividing the absorbed energy by the local absorption coefficient.

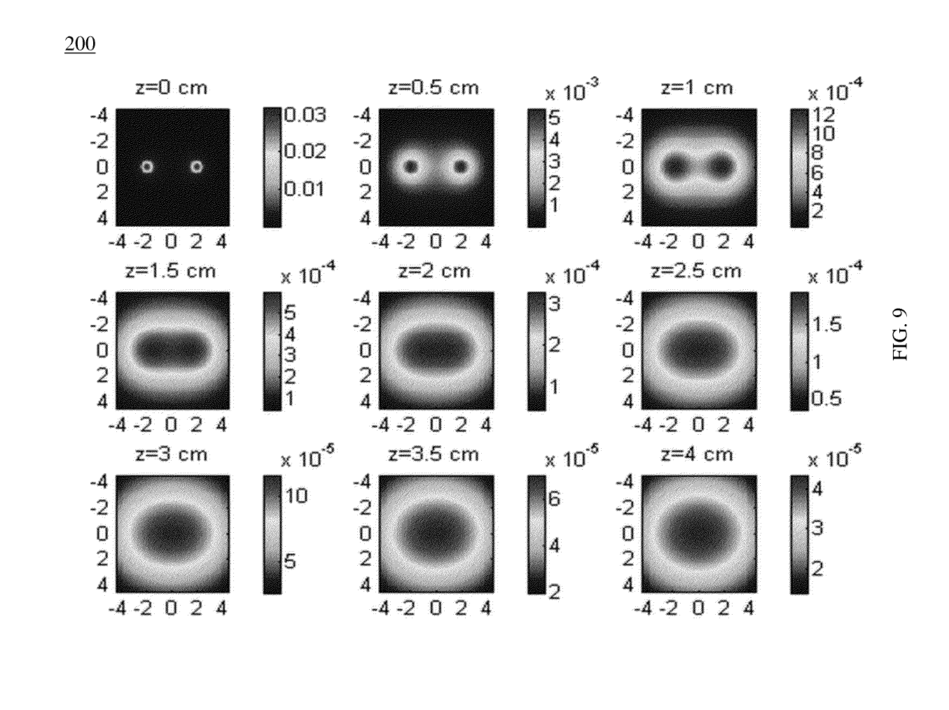

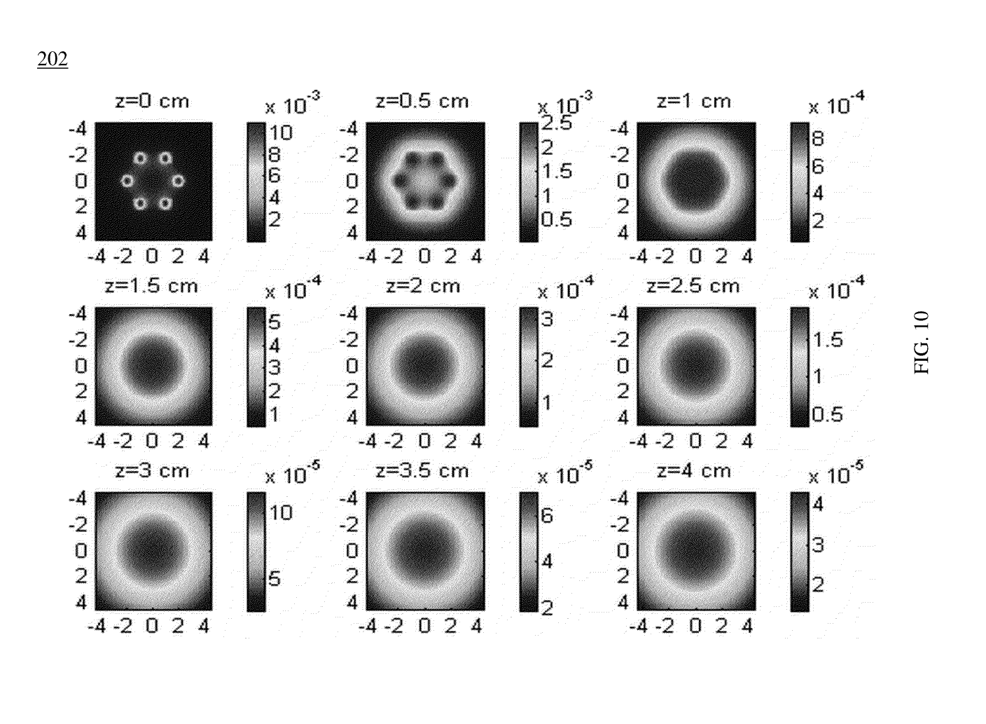

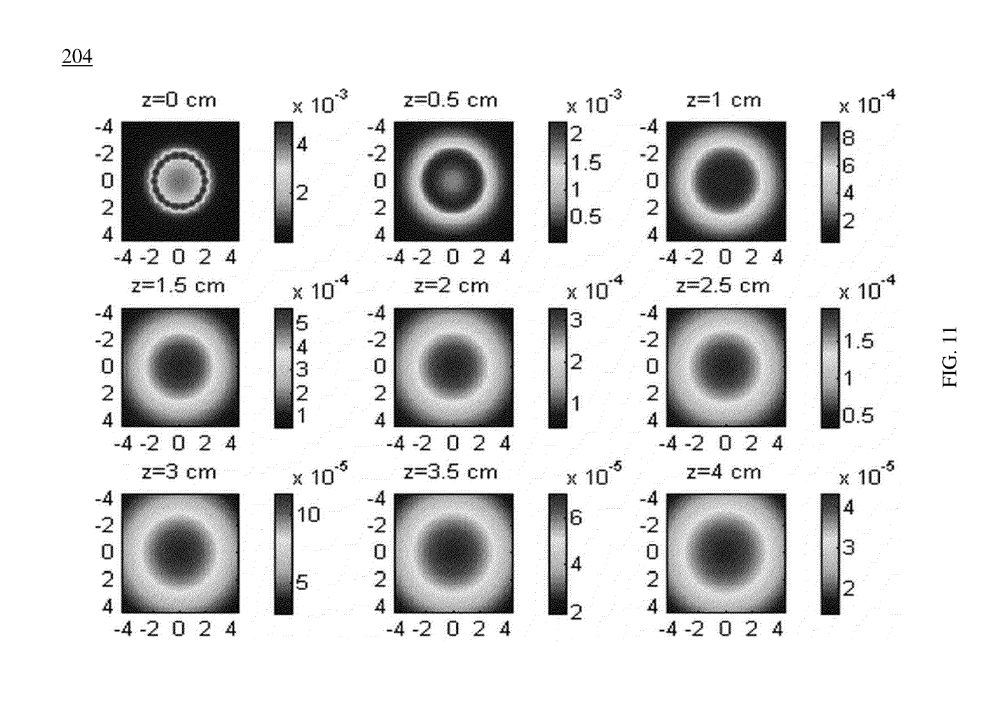

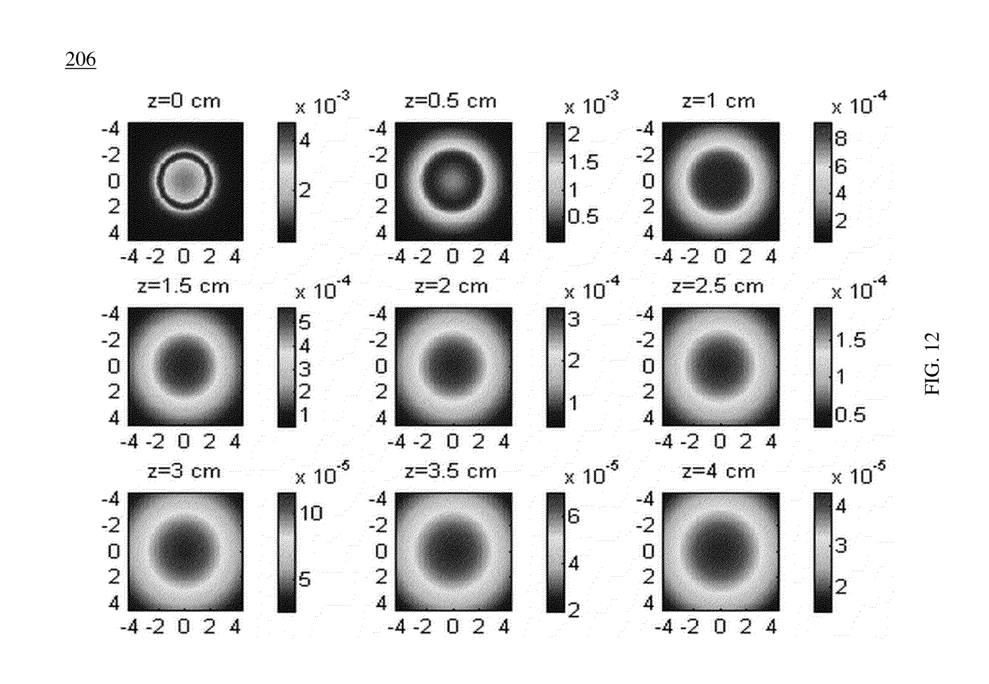

The results for the Monte Carlo simulations are shown in FIGS. 9, 10, 11, and 12. The simulated fluence distributions 200 (FIG. 9), 202 (FIG. 10), 204 (FIG. 11), and 206 (FIG. 12) are shown for optical fiber distributions respectively corresponding to distributions 100A (2 optical fibers), 102A (6 optical fibers), 104A (18 optical fibers), and 106 (36 optical fibers) of FIG. 7. The depth of illumination is labeled as “z” in FIGS. 9-12 and ranges from z=0 cm to z=4 cm, in 0.5-cm increments.

Although fluence distribution 200 (2 optical fibers) is asymmetric up to a 4 cm depth, the fluence distribution 200 varies from a bimodal fluence distribution at a depth from 0 cm to 1.5 cm but achieves a unimodal, elliptically-shaped distribution at a depth of 2.5 cm or greater. Furthermore, the distribution 202 for a 6-optical fiber endoscope approximates a symmetric fluence distribution at least at a depth of 1.5 cm or greater, as shown in FIG. 10. Additionally, the distribution 204 for an 18-optical fiber endoscope highly approximates a symmetric fluence distribution at a depth of at least 0.5 cm. The simulated for distributions 206 for a 36-optical fiber endoscope show similar results to the 18-optical fiber endoscope and exhibit a symmetrical fluence distribution even near the source (z=0 cm). Also, the attenuation of the 2-optical fiber distribution 200 occurs at greater rate with increasing thickness of model tissue than does a model endoscope with a greater number of optical fibers such as 8, 16, or 36 optical fibers having fluence distributions 202, 204, and 206 respectively shown in FIGS. 10, 11, and 12.

### Example 2

### Imaging in a Biomimetic System

Light from a Ti:sapphire laser (Symphotics TII, LS-2134) optically pumped by a Q-switched Nd:YAG (Symphotics-TII, LS-2122) passed through a convex lens and a 50/50 beam splitter that directed the light into the input surface of two optical fiber light splitters that were respectively coupled to optical fibers as shown in FIG. 8. The laser was tunable and delivered 20 ns pulses at a repetition rate of 15 Hz with an energy of 20 mJ/pulse at 750 nm Since two light splitters were used instead of one light splitter coupled to the plurality of optical fibers, the energy density was decreased on the input surface of the fibers by approximately half. Light from the 36 optical fibers in the endoscope diffusely propagated to an imaged target, which was immersed in an intralipid solution, having a reduced scattering coefficient of 4 cm⁻¹ and absorption coefficient 0.02 cm⁻¹.

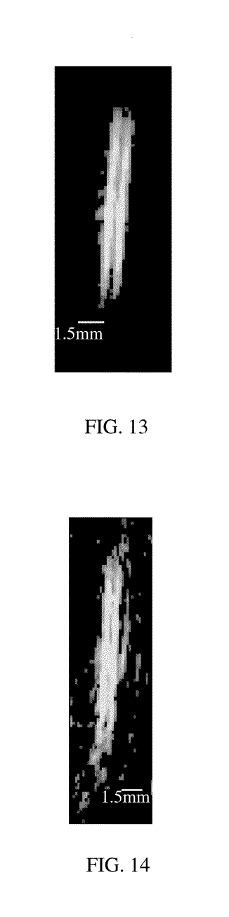

A biomimetic system was used to access the performance of the endoscope. The biomimetic system included a polyethylene tube filled with blood from a mouse that performed as a model for a blood vessel. The tube had inner and outer diameters of 0.86 mm and 1.27 mm, respectively. FIG. 13 shows a photoacoustic image of the tube filled with blood. The image is an average of 32 acquired image signals taken after a time delay from each corresponding laser pulse. The dynamic range for displaying the image was 15 decibels (dB). The image in FIG. 13 was acquired using only the intralipid solution between the tube and the probe. The maximum signal-to-noise ratio (SNR) for this image is 27, whereas the mean was 21. As used herein, “maximum SNR” refers to a greatest SNR obtained from among the 64 transducer channels, and the “mean SNR” refers to the average SNR from all 64 channels.

To mimic biological tissue that intercedes between an actual blood vessel and the endoscope, a 1 centimeter (cm) layer of chicken breast tissue was used to cover the tube filled with blood. Identical conditions were used to image this combination of chicken breast tissue and the tube as was used for the tube without the layer of tissue, and the resulting image is shown in FIG. 14. The tube is still visible with the addition of the layer of chicken breast tissue. The maximum SNR decreased to 22 as compared to the image in FIG. 13, and the mean SNR decreased to 14.

### Example 3

### Imaging Human Ovary

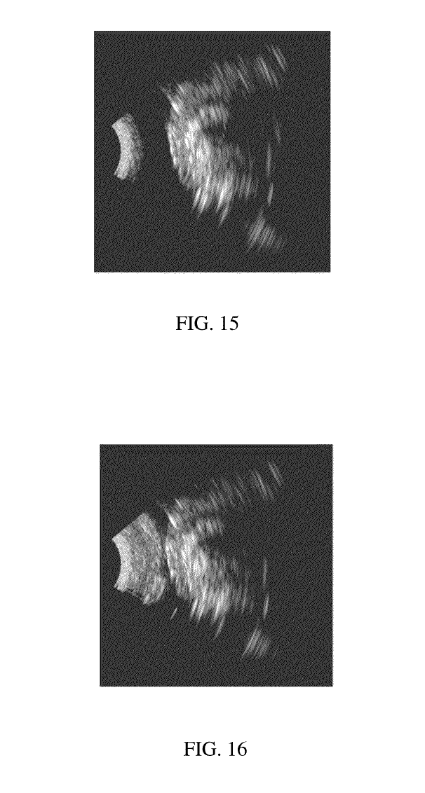

A human ovary was imaged ex vivo under conditions in Examples 1 and 2, using a depth of 1 cm below the endoscope. The ovary was mounted in an intralipid solution having a 4 cm⁻¹ scattering coefficient and a 0.02 cm⁻¹ absorption coefficient. FIG. 15 shows a co-registered image of the acquired ultrasound pulse-echo/photoacoustic frames of the human ovary. The grayscale image is the ultrasound pulse-echo, whereas the color image is derived from the photoacoustic signal. Vascularization of the ovary is clearly observed from the photoacoustic signal, and anatomical features also are visible from the ultrasound signal.

A 1 cm thick layer of chicken breast tissue was placed between the human ovary and the endoscope to simulate the tissue wall and muscle that would be present in a human subject. The acquired image of the breast tissue covered human ovary is shown in FIG. 16. Again, the anatomical and vascular features are discernible. Thus, the endoscope provides adequate resolution and sensitivity to probe human tissue with dual-modality, co-registry of images from such tissue.

The singular forms “a,” “an,” and “the” include plural referents unless the context clearly dictates otherwise. “Or” means “and/or.”

Various numerical ranges are disclosed in this patent application. Because these ranges are continuous, they include every value between the minimum and maximum values. The endpoints of all ranges reciting the same characteristic or component are independently combinable and inclusive of the recited endpoint.

All references are incorporated herein by reference.

While the invention has been described with reference to various embodiments, it will be understood by those skilled in the art that various changes can be made and equivalents can be substituted for elements thereof without departing from the scope of the invention. In addition, many modifications can be made to adapt a particular situation or material to the teachings of the invention without departing from the essential scope thereof. Therefore, it is intended that the invention not be limited to any particular embodiment disclosed for carrying out this invention, but that the invention will include all embodiments falling within the scope of the appended claims.

1. An endoscope comprising:
a sheath;
an ultrasound transducer disposed in the sheath to transmit an ultrasound frequency and to receive an image signal comprising an ultrasound signal and photoacoustic signal; and
a plurality of optical fibers interposed between the sheath and the ultrasound transducer to transmit light;
wherein the sheath comprises:
a first end configured to accept the ultrasound transducer and plurality of optical fibers; and
a second end to pass the ultrasound frequency and light out of the sheath.

2. The endoscope of claim 1, further comprising a coupling member to couple the sheath to the ultrasound transducer and the plurality of optical fibers.

3. The endoscope of claim 1, further comprising a handle disposed at the first end.

4. The endoscope of claim 3, wherein the handle and the ultrasound transducer are an integrated member.

5. The endoscope of claim 1, further comprising an optic disposed at the second end to transmit the light from the plurality of optical fibers.

6. The endoscope of claim 1, wherein the ultrasound transducer is disposed inside the sheath to terminate before an edge of the second end.

7. The endoscope of claim 1, wherein the plurality of optical fibers terminates from 1 mm to 15 mm before the second end of the sheath.

8. The endoscope of claim 1, wherein the endoscope is flexible such that the endoscope bends in response to an applied force.

9. The endoscope of claim 1, wherein the ultrasound transducer or plurality of optical fibers is immobilized in the sheath.

10. The endoscope of claim 1, wherein the ultrasound transducer or plurality of optical fibers has a rotary mobility, axial mobility, or a combination comprising at least one of the foregoing in the sheath.

11. The endoscope of claim 1, wherein an inner surface of the sheath comprises a coating effective to reflect the light from the plurality of optical fibers.

12. The endoscope of claim 11, wherein the coating comprises aluminum, silver, gold, platinum, copper, tin, tantalum, zinc, zirconium, silicon, an oxide thereof, or a combination comprising at least one of the foregoing.

13. The endoscope of claim 1, wherein the second end of the sheath comprises a notched structure configured to transmit the ultrasound frequency from the ultrasound transducer.

14. The endoscope of claim 1, wherein the sheath comprises a metal.

15. The endoscope of claim 1, wherein the plurality of optical fibers are perimetrically distributed about the ultrasound transducer.

16. The endoscope of claim 1, wherein the optical fibers are divided into at least two groups of optical fibers, the two groups being separated by at least a distance corresponding to a width traversed by an output of the ultrasound transducer.

17. The endoscope of claim 1, wherein the plurality of optical fibers includes more than 2 optical fibers.

18. The endoscope of claim 1, wherein each of the optical fibers of the plurality of optical fibers has a diameter from 50 μm to 1 mm.

19. The endoscope of claim 1, further comprising a film disposed on the plurality of optical fibers to prevent direct contact of the plurality of optical fibers with a tissue in use.

20. The endoscope of claim 1, wherein a fluence of the light transmitted by the plurality of optical fibers has a uniform illumination distribution at a distance from 4 mm to 40 mm beyond the second end of the endoscope.

21. The endoscope of claim 20, wherein the uniform illumination distribution is a Gaussian distribution.

22. The endoscope of claim 1, wherein the light has a power from 1 mJ/cm² to 24 mJ/cm².

23. The endoscope of claim 1, wherein the light has a wavelength from 600 nm to 1000 nm.

24. The endoscope of claim 1, wherein the light has a pulse length from 1 ns to 200 ns.

25. The endoscope of claim 1, wherein the light has a repetition rate from 1 Hz to several kHz.

26. The endoscope of claim 1, wherein the endoscope is a transvaginal probe, transrectal probe, transnasal probe, transesophageal probe, or transurethral probe.

27. The endoscope of claim 1, wherein a resolution of the endoscope is at least 0.2 mm.

28. A process of making an endoscope, the process comprising:
shaping a material to form a sheath;
inserting an ultrasound transducer into the sheath;
disposing a plurality of optical fibers into the sheath; and
coupling an end of the sheath to the ultrasound transducer to make the endoscope of claim 1.

29. A system for imaging comprising:
the endoscope of claim 1 comprising:
a sheath;
an ultrasound transducer disposed in the sheath to transmit an ultrasound frequency and to receive an image signal comprising an ultrasound signal and photoacoustic signal; and
a plurality of optical fibers interposed between the sheath and the ultrasound transducer to transmit light;
a near-infrared light source coupled to the plurality of optical fibers; and
an acquisition device to acquire an image signal from the ultrasound transducer, wherein the sheath comprises:
a first end configured to accept the ultrasound transducer and plurality of optical fibers; and
a second end to pass the ultrasound frequency and light out of the sheath.

30. The system of claim 29, further comprising an optical train to couple the near-infrared light source to the plurality of optical fibers.

31. The system of claim 30, wherein the optical train includes a lens to focus a light from the near-infrared light source onto an input end of the plurality of optical fibers.

32. The system of claim 30, wherein the optical train includes a beam splitter to split a light from the near-infrared light source into at least two light paths.

33. The system of claim 29, wherein the image signal comprises a photoacoustic signal and ultrasound signal.
